(19) United States
(12) Patent Application Publication
(10) Pub. No.: US 2026/0083652 A1
(43) Pub. Date: Mar. 26, 2026

(54) RENEWABLE ESTER EMOLLIENT FOR ENHANCED CUTANEOUS PERMEATION

(71) Applicant: Oxiteno S.A. Indústria e Comércio, São Paulo - SP (BR)

(72) Inventors: Alexandra Pezzolo PAVANELLI, São Bernardo - SP (BR); Andre Oliva DE PALMA, São Bernardo - SP (BR); Paula Maria NOGUEIRA AMBROGI, São Bernardo - SP (BR); Thiago Cagliumi ALVES, São Bernardo - SP (BR)

(73) Assignee: Oxiteno S.A. Indústria e Comércio, São Paulo - SP (BR)

(21) Appl. No.: 19/333,399

(22) Filed: Sep. 19, 2025

Related U.S. Application Data
(60) Provisional application No. 63/697,109, filed on Sep. 20, 2024.

(51) Int. Cl. A61K 8/37; A61Q 5/12

(52) U.S. Cl. CPC A61K 8/37; A61Q 5/12; A61K 2800/48; A61K 2800/49; A61K 2800/522; A61K 2800/5922

(57) ABSTRACT

The present disclosure provides a personal care formulation including an ester emollient, which may be derived from an alcohol and a fatty acid. A method of moisturizing skin or hair comprises applying an effective amount of the personal care formulation. A process for preparing the personal care formulation includes preparing the ester emollient by esterification reaction of alcohol and fatty acid.

### RELATED APPLICATION(S)

This application is a NON-Provisional patent application which claims the benefit of priority under 35 USC § 119(e) of U.S. Provisional Patent Application No. 63/697,109 filed on Sep. 20, 2024. The contents of the above application are all incorporated by reference as if fully set forth herein in their entirety.

### FIELD AND BACKGROUND OF THE INVENTION

The present disclosure relates to the field of personal care formulations, particularly to emollients for use in skincare and hair care products. More specifically, the disclosure provides a novel renewable ester emollient derived from natural sources, which can be used in personal care formulations to enhance the cutaneous permeation of active ingredients. The disclosure also relates to methods of preparing such formulations and their use in moisturizing skin or hair.

Within the raw materials used in personal care products, emollients may be key ingredients in many formulations, playing an important role in performance (i.e., moisturizing) and sensory attributes (e.g., texture, spreadability, oily feel, etc.).

Emollients may have different chemical structures, among which esters may be one of the classes. These ingredients may have the ability to remain on the skin's surface, act as lubricants, reduce flaking and improve appearance, decrease water loss, retain moisture, improve the penetration of cosmetics into the skin, and boost the texture of the formulation.

Emollients may be found in products that offer moisturizing properties, such as those for body care (body creams, lotions, deodorants, etc.), hair care, and sun protection. In addition to all these benefits, some emollients can also act as UV filter solubilizers in sunscreen formulations.

Mostly due to cost constraints, most emollients used today may be derived from petrochemical chemicals. The largest portion of the market may be represented by emollients, such as mineral oils, silicones, and petroleum-derived esters, such as isopropyl palmitate and isopropyl myristate.

In the personal care market, there may be a growing demand for natural ingredients, especially those derived from plant and renewable sources. Consumer awareness about the environmental impacts of products may have significantly increased, so companies should strive to provide high-performing, environmentally responsible products. In some aspects, the increasing interest in natural ingredients may present opportunities for developing new raw materials. However, this trend may also introduce challenges for researchers and formulators tasked with delivering products containing novel renewable ingredients while improving performance. Since esters can be obtained from sustainable, renewable raw materials, green-esters may be increasingly gaining share in cosmetic formulations.

### SUMMARY OF THE INVENTION

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a personal care formulation is provided. The personal care formulation includes a mixture of a first moisturizing agent, a second moisturizing agent, and a cosmetically acceptable vehicle. The first moisturizing agent has a formula R—COO—C₅H₁₁, wherein R is a linear or branched alkyl group of formula —(CH₂)ₙ—CH₃ and n is 6. The second moisturizing agent has a formula R—COO—C₅H₁₁, wherein R is a linear or branched alkyl group of formula —(CH₂)ₙ—CH₃, where n is 8.

According to other aspects of the present disclosure, the personal care formulation may include one or more of the following features. The personal care formulation may further comprise retinyl acetate. The first moisturizing agent may comprise isoamyl caprylate. The second moisturizing agent may comprise isoamyl caprate. The first moisturizing agent may be about 0.5% to about 15% by weight of the personal care formulation. The second moisturizing agent may be about 0.5% to about 15% by weight of the personal care formulation. The personal care formulation may further comprise at least one additive selected from the group consisting of thickeners, emulsifiers, emollients, chelating agents, preservatives, antioxidants, pH adjusters, and combinations thereof.

According to another aspect of the present disclosure, a method of moisturizing skin or hair is provided. The method includes applying to skin or hair an effective amount of a personal care formulation comprising a mixture of a first moisturizing agent and a second moisturizing agent, and a cosmetically acceptable vehicle. The first moisturizing agent has a formula R—COO—C₅H₁₁, wherein R is a linear or branched alkyl group of formula —(CH₂)ₙ—CH₃ and n is 6. The second moisturizing agent has a formula R—COO—C₅H₁₁, wherein R is a linear or branched alkyl group of formula —(CH₂)ₙ—CH₃, where n is 8.

According to other aspects of the present disclosure, the method may include one or more of the following features. The personal care formulation may further comprise retinyl acetate. The first moisturizing agent may comprise isoamyl caprylate. The second moisturizing agent may comprise isoamyl caprate. The first moisturizing agent may be about 0.5% to about 15% by weight of the personal care formulation. The second moisturizing agent may be about 0.5% to about 15% by weight of the personal care formulation. The personal care formulation may further comprise at least one additive selected from the group consisting of thickeners, emulsifiers, emollients, chelating agents, preservatives, antioxidants, pH adjusters, and combinations thereof.

According to another aspect of the present disclosure, a process for preparing a personal care formulation is provided. The process includes preparing a mixture of a first moisturizing agent and a second moisturizing agent by an esterification reaction of alcohol derived from sugar cane and fatty acid derived from a vegetable source. The first moisturizing agent has a formula R—COO—C₅H₁₁, wherein R is a linear or branched alkyl group of formula —(CH₂)ₙ—CH₃ and n is 6. The second moisturizing agent has a formula R—COO—C₅H₁₁, wherein R is a linear or branched alkyl group of formula —(CH₂)ₙ—CH₃, where n is 8. The process further includes combining the mixture of the first moisturizing agent and the second moisturizing agent with a cosmetically acceptable vehicle.

According to other aspects of the present disclosure, the process may include one or more of the following features. The personal care formulation may further comprise retinyl acetate. The first moisturizing agent may comprise isoamyl caprylate. The second moisturizing agent may comprise isoamyl caprate. The first moisturizing agent may be about 0.5% to about 15% by weight of the personal care formulation, and the second moisturizing agent may be about 0.5% to about 15% by weight of the personal care formulation. The personal care formulation may further comprise at least one additive selected from the group consisting of thickeners, emulsifiers, emollients, chelating agents, preservatives, antioxidants, pH adjusters, and combinations thereof.

The foregoing general description of the illustrative embodiments and the following detailed description thereof are merely exemplary aspects of the teachings of this disclosure and are not restrictive.

### BRIEF DESCRIPTION OF THE SEVERAL VIEWS OF THE DRAWING(S)

The disclosure can be understood in consideration of the following detailed description of various embodiments in connection with the accompanying drawings. Non-limiting and non-exhaustive examples are described with reference to the following figures.

FIG. 1 illustrates a cross-section of the epidermis, according to aspects of the present disclosure.

FIG. 2 depicts potential pathways of a topical formulation through the stratum corneum, according to an embodiment.

FIG. 3 depicts a flowchart for creating a moisturizing composition, according to aspects of the present disclosure.

FIG. 4 illustrates a flowchart for applying a personal care formulation, according to an embodiment.

FIG. 5A shows stages of cell growth in a laboratory culture dish, according to aspects of the present disclosure.

FIG. 5B illustrates a microscopic cross-section of human skin tissue, according to an embodiment.

FIG. 6A shows a multi-well plate containing liquid samples, according to an embodiment.

FIG. 6B illustrates a multi-well plate with a sample being placed, according to aspects of the present disclosure.

FIG. 7A depicts a block diagram of an experimental design system for evaluating emollients, according to an embodiment.

FIG. 7B shows an experimental design for comparing retinyl acetate formulations, according to aspects of the present disclosure.

FIG. 8 illustrates a bar graph comparing emollient permeation on reconstructed human epidermis, according to an embodiment.

FIG. 9 depicts a graph showing accumulated retinyl acetate over time, according to aspects of the present disclosure.

FIG. 10 shows a bar graph comparing retinyl acetate concentration on reconstructed human epidermis, according to aspects of the present disclosure.

### DESCRIPTION OF SPECIFIC EMBODIMENTS OF THE INVENTION

For purposes of promoting an understanding of the principles of the disclosure, reference will now be made to the embodiments illustrated in the drawings, and specific language will be used to describe the same. It will nonetheless be understood that no limitation of the scope of the disclosure is intended by the illustration and description of certain embodiments of the disclosure. In addition, any alterations and/or modifications of the illustrated and/or described embodiment(s) are contemplated as being within the scope of the present disclosure. Further, any other applications of the principles of the disclosure, as illustrated and/or described herein, as would normally occur to one skilled in the art to which the disclosure pertains, are contemplated as being within the scope of the present disclosure.

The following description sets forth exemplary aspects of the present disclosure. It should be recognized, however, that such description is not intended as a limitation on the scope of the present disclosure. Rather, the description also encompasses combinations and modifications to those exemplary aspects described herein.

A number of implementations have been described. Nevertheless, it will be understood that various modifications may be made without departing from the spirit and scope of the disclosure. Accordingly, other implementations are within the scope of the following claims.

The present disclosure provides a moisturizing agent (e.g., emollient) for enhancing the permeation of an active ingredient in a personal care formulation. The emollient may include an ester. The ester, which may belong to the Oxismooth® line (e.g., Oxismooth® CO, Oxismooth® CP, Oxismooth® ST), may be significantly more sustainable compared to those already on the market and may be derived from 100% natural and renewable sources. The line is made from 100% renewable sources: isoamyl alcohol from sugarcane, and other oleochemical derivatives to create innovative and sustainable formulations. Certified by the COSMOS-standard, the line is not phototoxic or cytotoxic. Its properties also allow the replacement of silicones in different formulations, contributing to the sustainability of the end product. The Oxismooth® line of esters was developed based on the company's (e.g., Indorama Oxiteno, which is part of Indorama Ventures) Greenformance concept, which associates sustainability and performance. The Greenformance concept is based on three key pillars: 1. Use of renewable resources (natural raw materials in substitution of synthetics and petrochemicals), 2. Environmental care (biodegradable and concentrated products to reduce energy, water and packaging consumption), and 3. health and wellness (high purity and performance formulas, mild and non-irritant).

The OXISMOOTH® line comprises three different emollient esters. Each of them (OXISMOOTH® CO, OXISMOOTH® CP, and OXISMOOTH® ST) has been designed to provide different sensory profiles in a wide range of applications, including creams and lotions, hair treatment, deodorant, baby care, hair dyes, etc. The general benefits of these solutions are 100% renewable source, COSMOS-ECOCERT certified, moisturizing, conditioning properties, high spreadability, fast absorption, pleasant sensory feel, non-greasy (e.g., not oil), preservative-free, easy handling, incorporation (e.g., cold incorporation), suitable (e.g., high compatibility) for all cosmetic formulations.

OXISMOOTH® CO is a natural-based conditioning agent that replaces silicone in skin and hair formulations.

OXISMOOTH® CP is a green emollient with high spreadability and fast absorption.

OXISMOOTH® ST is a green emollient with high spreadability, low tack, and an excellent alternative to mineral oils.

Any of the esters may be incorporated into personal care formulations, such as skin care, hair, male cosmetic, and baby care products, where it may serve multiple functions. The ester may act as a moisturizing agent or an emollient, providing moisturizing and texture-improving properties, while also enhancing the cutaneous permeation of active ingredients (e.g., retinyl acetate) in the formulation. This dual functionality may allow for improved efficacy of personal care (e.g., skincare) formulations, potentially enabling lower concentrations of active ingredients to be used. Furthermore, the use of the ester may align with the growing consumer demand for natural and environmentally responsible ingredients in personal care products, offering a sustainable alternative to traditional petrochemical-derived emollients. On top of the environmental nature of the molecules per se, the production process may be optimized to minimize loss and waste generation as well, yielding a fully optimized supply chain and product lifecycle. In some embodiments, the disclosure also encompasses methods for preparing personal care formulations incorporating the ester, as well as methods of using such formulations for moisturizing skin or hair.

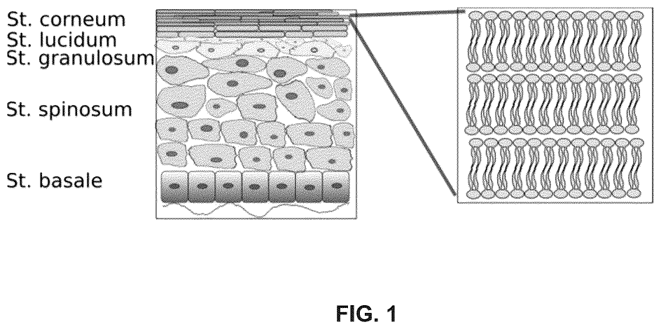

The skin (e.g., human skin) is a complex organ that acts as the first protecting barrier of the body. The skin is a multilayer tissue, and one of its functions is to guard the body against external circumstances by functioning as an effective barrier to the absorption of exogenous particles. The skin may include three main layers, such as the epidermis, dermis, and subcutaneous tissue (e.g., hypoderms). The dermis is a vascularized tissue (e.g., contains blood vessels). The dermis may be hydrophilic. The subcutaneous tissue may include adipocytes. The subcutaneous tissue may be a hydrophilic layer. FIG. 1 illustrates the structure of the epidermis, which is relevant to skin permeation. The epidermis, the outermost layer of the skin, may be divided into five layers: the stratum corneum, stratum lucidum, stratum granulosum, stratum spinosum, and stratum basale. The epidermis is not a vascularized tissue (e.g., lacks blood vessels). The epidermis may include non-viable tissue (stratum corneum) and viable tissue (stratum lucidum, stratum granulosum, stratum spinosum, and stratum basale). Non-viable tissue may be a lipophilic layer and may be 15% water. Viable tissue may be a hydrophilic layer and may be 70% water. The stratum corneum, the outermost layer, is depicted as multiple layers of flattened cells. An enlarged inset of the stratum corneum shows the detailed structure of its lipid bilayers, which are crucial for the skin's barrier function. This detailed representation of the skin's structure is important for understanding how the emollient and active ingredients in the personal care composition might interact with and penetrate through the different layers of the epidermis.

The stratum corneum may act as a critical part in the barrier function for topical product penetration. The intercellular lipid matrix may include a mixture of fatty acids, ceramides, cholesterol, cholesterol esters, and a small fraction of cholesterol sulfate. Considering this composition and its water resistance, the stratum corneum is considered the main layer to be crossed to allow product permeation through the skin.

In some aspects, the personal care composition may be formulated to target specific layers of the epidermis. For example, certain active ingredients may be more effective when delivered to the stratum basale, where new skin cells are produced, while others may be designed to act on the stratum corneum to improve the skin's surface appearance and barrier function. The composition may be formulated to optimize the penetration of the active ingredients and the emollient through the stratum corneum and into the deeper layers of the epidermis.

In some cases, the composition may include additional ingredients that enhance the permeation of the active ingredients and the emollient through the stratum corneum. These may include penetration enhancers, solvents, or other ingredients known in the art for improving skin permeation. The composition may also include ingredients that help to maintain the integrity of the stratum corneum's lipid bilayers, such as ceramides, cholesterol, or fatty acids, which can further enhance the delivery of the active ingredients and the emollient to the target layers of the epidermis.

In some embodiments, the composition may provide a sustained release of the active ingredients and the emollient over time. This can be achieved, for example, by encapsulating the active ingredients and the emollient in microspheres, liposomes, or other delivery systems that gradually release the ingredients into the skin. This can provide prolonged moisturization and treatment effects and can also reduce the frequency of application needed.

In some cases, the composition may be formulated to minimize irritation or sensitivity reactions that can sometimes occur with topical applications. This can be particularly important when the composition includes active ingredients that are known to cause skin irritation, such as certain retinoids or acids. The composition may include soothing or anti-inflammatory ingredients to counteract any potential irritation, and the emollient itself may also have soothing or anti-inflammatory properties.

In some aspects, the composition may be formulated for specific skin types or conditions. For example, the composition may be formulated for dry skin, oily skin, sensitive skin, acne-prone skin, aged skin, or other skin types or conditions. The composition may include additional ingredients that are beneficial for the specific skin type or condition, and the amount or type of emollient and active ingredients may be adjusted accordingly.

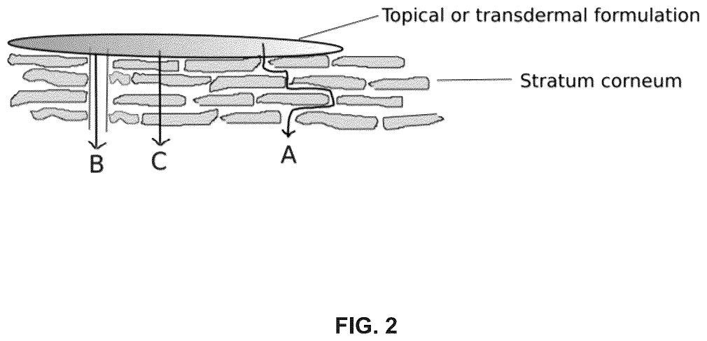

FIG. 2 illustrates the three potential pathways for a topical or transdermal formulation to penetrate through the stratum corneum, the outermost layer of the skin. These pathways include intercellular penetration, follicular penetration, and transcellular penetration.

In some aspects, the intercellular penetration pathway, represented by arrow A in FIG. 2, involves the movement of the formulation between the skin cells of the stratum corneum. This pathway may be particularly relevant for the delivery of personal care formulation containing one or more moisturizing agents. The lipophilic nature of these agents may facilitate their movement through the lipid-rich environment of the intercellular spaces.

In some cases, the follicular penetration pathway, represented by arrow B in FIG. 2, involves the movement of the formulation through hair follicles present in the skin. This pathway may be particularly relevant for formulations applied to areas of the skin with a high density of hair follicles, such as the scalp or body. The formulation may penetrate the hair follicles and then diffuse into the surrounding skin tissue.

In some embodiments, the transcellular penetration pathway, represented by arrow C in FIG. 2, involves the movement of the formulation directly through the skin cells of the stratum corneum. This pathway may be particularly relevant for smaller molecules in the formulation that can pass through the cell membranes.

In some aspects, the personal care formulation may optimize the penetration of the moisturizing agent and/or active ingredient through one or more of these pathways. For example, the formulation may include ingredients that enhance intercellular penetration, such as penetration enhancers or solvents. Alternatively, the formulation may enhance follicular penetration, for example by including ingredients that increase the solubility of the agents in sebum, the oily substance present in hair follicles. In some cases, the formulation may enhance transcellular penetration, for example, by including ingredients that increase the permeability of skin cell membranes.

In some embodiments, the personal care formulation may utilize multiple penetration pathways simultaneously. For example, the formulation may include a combination of ingredients that enhance both intercellular and follicular penetration. This could potentially increase the overall penetration of the moisturizing agent into the skin, thereby enhancing the moisturizing effect of the formulation.

The examples demonstrate cutaneous permeation of the green emollient through the epidermis. Cutaneous permeation may be characterized by dermal penetration and dermal permeation. Dermal penetration may be described as the movement of a chemical from the outer surface of the skin into the epidermis but not necessarily into the circulatory system. Dermal permeation may be described as the penetration through one layer of the skin into another, which is both functionally and structurally different from the first layer.

Dermal penetration is a consideration in the development of topical personal care formulations, as it can determine how effectively the active ingredient(s) and moisturizing agent(s) can enter the skin layers. Dermal penetration may be influenced by various factors, including the molecular size, lipophilicity, and chemical structure of the compounds in the formulation. The ingredient(s) of the personal care formulation may achieve optimal dermal penetration, allowing them to effectively moisturize and nourish the skin without necessarily entering the bloodstream. The targeted penetration can help maximize the efficacy of the personal care formulation while minimizing potential systemic effects.

The dermal permeation process involves the movement of compounds through various layers of the skin, including the stratum corneum, viable epidermis, and dermis. Each of these layers presents different barriers and challenges for permeation. The ability of a compound to permeate through these layers depends on factors such as its molecular weight, lipophilicity, and the formulation in which it is delivered. Dermal permeation may be crucial for the effectiveness of topical formulations, as it can allow active ingredients to reach their intended target sites within the skin. Dermal permeation may be particularly important for the moisturizing agent to effectively hydrate and nourish the skin. The synergistic combination of the ingredients of the personal care formulation may enhance dermal permeation. The enhanced permeation could lead to improved moisturization of the skin, as the active ingredients are able to reach deeper layers where they can provide more effective and long-lasting hydration.

The personal care formulation may include one or more ingredients. In some examples, a personal care formulation may include a moisturizing agent. In some examples, a personal care formulation may include a moisturizing agent and an active ingredient. In some examples, a personal care formulation may include a moisturizing agent, an active ingredient, and one or more additives. The moisturizing agent may be an emollient. The emollient may include an ester. The additive may include a cosmetically acceptable vehicle. The additive may include additional moisturizing agents. In some examples, a personal care formulation may include a first moisturizing agent and a second moisturizing agent. In some examples, a personal care formulation may include a first moisturizing agent, a second moisturizing agent, and an active ingredient. In some embodiments, a person care formulation may include a first moisturizing agent, a second moisturizing agent, an active ingredient, and a cosmetically acceptable vehicle.

The ester may be synthesized from an alcohol (e.g., amyl alcohol, isoamyl alcohol, etc.) and a fatty acid (e.g., vegetal oil). The alcohol (e.g., amyl alcohol, isoamyl alcohol, etc.) may be derived from sugar cane, which may be a widely-available natural raw material in Brazil. The fatty acids may be derived from a vegetable source, such as coconut or palm kernel. The fatty acid may be obtained from an environmentally responsible source. Distinct fatty acid chain length cuts may be used for producing these emollients, providing them with different sensory properties (e.g., spreadability and feeling of greasiness), enabling formulators to design innovative and sustainable formulations for the personal care market.

Amyl alcohol, also known as pentanol, is a class of organic compounds with the general formula C5H11OH. Isoamyl alcohol, a specific isomer of amyl alcohol with the formula (CH3)2CHCH2CH2OH, may be used as a reactant in the synthesis of the moisturizing agent. Isoamyl alcohol, also known as isopentyl alcohol or 3-methyl-1-butanol, is a branched five-carbon alcohol with the chemical formula (CH3)2CHCH2CH2OH. Isoamyl alcohol may be a reactant in the esterification reaction to produce the moisturizing agent. Isoamyl alcohol may be derived from sugar cane through fermentation and distillation processes. The properties of isoamyl alcohol can vary based on its source and the specific isomer involved. Isoamyl alcohol generally refers to one of several isomers of pentyl alcohol with the formula C₅H₁₂O. The differences in properties are influenced by the position of the hydroxyl group (—OH) and the branching of the carbon chain. Common isomers of isoamyl alcohol include isoamyl alcohol (3-methyl-1-butanol), 2-methyl-1-butanol, and 1 pentanol. Properties affected by isomerism include boiling and melting points, solubility, odor and flavor, reactivity, and uses in different applications.

The renewable source of isoamyl alcohol (e.g., from sugar cane) contributes to the sustainable nature of the resulting ester emollient, aligning with the growing demand for natural and environmentally responsible ingredients in personal care products. The use of sugar cane-derived isoamyl alcohol distinguishes it from synthetic or petroleum-derived alternatives, emphasizing the commitment to renewable resources in the formulation of the personal care product. Sugar cane may be a widely available natural raw material, particularly in Brazil. Sugar-cane derived isoamyl alcohol may impact the final properties of the moisturizing agent, including its molecular structure, polarity, and potential interactions with skin or hair.

The fatty acid used in the synthesis of the moisturizing agent may be derived from vegetable sources, such as coconut or palm kernel oil. Coconut oil may be derived from the meat of mature coconuts harvested from the coconut palm (Cocos nucifera), while palm kernel oil may be extracted from the kernel or seed of the oil palm fruit (Elaeis guineensis). These fatty acids are typically octanoic (caprylic) acid or decanoic (capric) acid, which correspond to chain lengths of 8 and 10 carbon atoms, respectively. The use of these medium-chain fatty acids contributes to the unique properties of the resulting ester emollient, including its spreadability, skin feel, and moisturizing capabilities. The choice of vegetable-derived fatty acids aligns with the goal of creating a sustainable, renewable ingredient for personal care formulations. The specific fatty acid used can impact the final properties of the moisturizing agent, such as its melting point, viscosity, and skin penetration characteristics. By utilizing fatty acids from environmentally responsible sources, the formulation further enhances its eco-friendly profile while maintaining high performance as an emollient in skincare applications.

The ester may have a formula R—C(═O)—O—R₁, where R—C(═O) represents an acyl group, R may be an alkyl or aryl group, and R₁ represents an alkyl group. R—C(═O) includes a carbonyl group (C═O) bonded to an alkyl or aryl group. Radicals of R or R₁ are not typically part of the stable ester structure but may be involved in the formation or reaction processes related to the ester. In some examples, R is a linear or branched alkyl group, and R₁ is a linear or branched alkyl group having less than or equal to 6 carbons. In some examples, R is a linear or branched alkyl group having 7 carbons, and R₁ is a linear or branched alkyl group having less than or equal to 6 carbons. In some examples, R is a linear or branched alkyl group having 9 carbons, and R₁ is a linear or branched alkyl group having less than or equal to 6 carbons. In some examples, R is a linear or branched alkyl group, and R₁ is a linear or branched alkyl group having 5 carbons. In some examples, R is a linear or branched alkyl group having 7 carbons, and R₁ is a linear or branched alkyl group having 5 carbons. In some examples, R is a linear or branched alkyl group having 9 carbons, and R₁ is a linear or branched alkyl group having 5 carbons. In some examples, R is a linear or branched alkyl group of the formula —(CH₂)ₙ—CH₃, where n is 6 to 12, and R₁ is a branched alkyl group. In some examples, R is a linear or branched alkyl group of the formula —(CH₂)ₙ—CH₃, where n is 6, 7, 11, and 12, and R₁ is a branched alkyl group

An acyl group R is a functional group derived from a carboxylic acid by removing the hydroxyl (—OH) group. The acyl group contributes to the overall structure and properties of the ester, influencing characteristics such as its melting point, viscosity, and skin feel. The specific acyl group used can affect the emollient properties of the resulting ester, including its spreadability and moisturizing efficacy when incorporated into personal care formulations. The alkyl group R₁ may be derived from an alcohol or may be an alcohol residue. The specific structure of the alkyl group R₁ influences properties such as the melting point, viscosity, and skin penetration characteristics of the resulting moisturizing agent. For example, a longer alkyl chain may increase the emollient properties of the moisturizing agent, while a branched chain may affect its spreadability on the skin. The choice of alkyl radical length and structure in the moisturizing agent can be tailored to optimize the desired properties of the personal care formulation.

The ester may have a formula R—COO—C₅H₁₁, wherein R is a linear or branched alkyl group of formula —(CH₂)ₙ—CH₃ and n is a natural number. The ester may have a formula R—COO—C₅H₁₁, wherein R is a linear or branched alkyl group of formula —(CH₂)ₙ—CH₃ and n is 6. The ester may have a formula R—COO—C₅H₁₁, wherein R is a linear or branched alkyl group of formula —(CH₂)ₙ—CH₃ and n is 8.

The ester may have a formula R—COO—CH₂CH₂CH(CH₃)₂, where R is a linear or branched alkyl group of formula —(CH₂)ₙ—CH₃ and n is a natural number. In the context of the ester formula R—COO—CH₂CH₂CH(CH₃)₂, the acyl group is represented by R—C(═O)—, where R is the alkyl group derived from the fatty acid used in the esterification reaction. In some examples, the ester may have a formula R—COO—CH₂CH₂CH(CH₃)₂, where R is a linear or branched alkyl group of formula —(CH₂)ₙ—CH₃ and n is 6 to 8. In some examples, the ester may have a formula CH3-(CH₂)₆—COO—CH₂CH₂CH(CH₃)₂. In some examples, the ester may be isoamyl octanoate (isoamyl caprylate). In some examples, the ester may have a formula CH3-(CH₂)₈—COO—CH₂CH₂CH(CH₃)₂. In some examples, the ester may be isoamyl decanoate (isoamyl caprate).

In the moisturizing agent described, the acyl group is derived from either octanoic (caprylic) acid or decanoic (capric) acid, depending on whether n is 6 or 8 in the formula —(CH₂)ₙ—CH₃. In this case, the fatty acid is typically either octanoic (caprylic) acid when n is 6, or decanoic (capric) acid when n is 8. These medium-chain fatty acids are chosen for their specific properties that contribute to the emollient and moisturizing capabilities of the resulting ester. The choice of alkyl group length can affect various properties of the moisturizing agent, such as its melting point, viscosity, and skin penetration characteristics, which in turn influence the performance and feel of the personal care formulation.

The ester may have a formula R—COO—CH₂CH₂CH(R₁)₂R₂ where R is a linear or branched alkyl group of the formula —(CH₂)ₙ—CH₃, with n between 1 to 12, R₁ is independently, H, C₁-C₁₀ alkyl, or a substituted alkyl group, and an R₂ is an unsubstituted linear or branched C₁-C₁₂ alkyl group. In some cases, the two R₁ groups may be attached to the same carbon, forming a branched structure. In some cases, the total number of carbon atoms in the acyl portion (R—CO—) is 4 to 9. In some cases, the total number of carbon atoms in the acyl group is 8 carbons. In some cases, the total number of carbons in the acyl group is 10 carbons.

In some cases, R is a linear alkyl group of the formula —(CH₂)ₙ—CH₃, with n between 2 and 7 (i.e., 3-8 carbons total for R) and/or, R₁ is independently, H or C₁-C₃ alkyl, and/or R₂ is an unsubstituted linear or branched C₁-C₆ alkyl group. In some cases, the two R₁ groups may be attached to the same carbon, forming a branched structure. In some cases, the total number of carbon atoms in the acyl portion (R—CO—) is 4 to 9.

In some cases, R is a linear alkyl group of the formula —(CH₂)ₙ—CH₃, with n between 2 and 7 (i.e., 3-8 carbons total for R) and/or, R₁ is independently, H or C₁-C₃ alkyl, and/or R₂ is an unsubstituted linear or branched C₁-C₆ alkyl group. In some cases, the two R₁ groups may be attached to the same carbon, forming a branched structure.

In some cases, R is a linear alkyl group of the formula —(CH₂)ₙ—CH₃, with n between 1 and 12 (i.e., 2-13 carbons total for R) and/or, R₁ is independently, H or C₁-C₅ alkyl with two R₁ groups attached to the same carbon, forming a branched structure and/or R₂ is an unsubstituted linear or branched C₁-C₈ alkyl group.

In some cases, R is a linear alkyl group of the formula —(CH₂)ₙ—CH₃, with n between 1 and 12 (i.e., 2-13 carbons total for R) and/or, R₁ is independently, H or C₁-C₈ alkyl with two R₁ groups attached to the same carbon, forming a branched structure and/or R₂ is an unsubstituted linear or branched C₁-C₁₀ alkyl group.

In some cases, R₁ is a substituted alkyl group with a single substituent (e.g., methyl group, hydroxyl group, etc.). Alkyl groups are hydrocarbon chains derived from alkanes, typically represented as R or R₁, with structures like —CH₃ (methyl), —CH₂CH₃ (ethyl), —CH₂CH₂CH₃ (propyl), and so on. Substituted alkyl groups are alkyl groups that have one or more atoms or groups attached to the main carbon chain, which modifies the basic structure. Single substituent means that only one additional group or atom is attached to the main alkyl chain.

In some examples, the personal care formulation may include a mixture of a first moisturizing agent and a second moisturizing agent, which are different compounds. The different compounds may be based on different formula or same formula with different variables. In some examples, the personal care formulation may include a mixture of a first moisturizing agent and a second moisturizing agent in which each of the first moisturizing agent and the second moisturizing agent has a formula R—COO—CH₂CH₂CH(CH₃)₂, where R is a linear or branched alkyl group of formula —(CH₂)ₙ—CH₃ and n is a natural number, and the first moisturizing agent has a different n than the second moisturizing agent. In some examples, the personal care formulation may include a mixture of a first moisturizing agent and a second moisturizing agent, where each of the first moisturizing agent and the second moisturizing agent having a formula R—COO—CH₂CH₂CH(CH₃)₂, R is a linear or branched alkyl group of formula —(CH₂)ₙ—CH₃, and n is a natural number, and n is 6 for the first moisturizing agent and n is 8 for the second moisturizing agent.

In some aspects, the personal care formulation may include a moisturizing agent of formula R—COO—CH₂CH₂CH(CH₃)₂, where R is a linear or branched alkyl group of formula —(CH2)ₙ—CH3. In this formula, n may be an integer from 6 to 8, indicating that the alkyl chain can vary in length from 7 to 9 carbon atoms in total. In some cases, n may be 6 or 8, corresponding to the use of octanoic (caprylic) acid or decanoic (capric) acid, respectively, in the synthesis of the moisturizing agent. The specific value of n may be selected based on various factors, such as the desired properties of the moisturizing agent, the available sources of fatty acids, and the specific requirements of the personal care formulation.

In some embodiments, the moisturizing agent may be present in about 0.5% to about 15% by weight of the personal care formulation, such as 5% by weight. The specific concentration of the moisturizing agent in the formulation may be adjusted based on various factors, such as the desired moisturizing effect, the stability of the formulation, the sensory properties of the formulation, or other characteristics. For example, a higher concentration of the moisturizing agent may provide a stronger moisturizing effect, while a lower concentration may result in a lighter, less greasy feel.

In some cases, the personal care formulation may also include at least one additional moisturizing agent. The additional moisturizing agent may be any suitable agent for providing moisturizing benefits to the skin or hair. Non-limiting examples of additional moisturizing agents include isoamyl caprylate, isoamyl caprate, caprylic triglyceride, capric triglyceride, glycerol, urea, hydroxyalkylurea, lactic acid, sodium lactate, and sodium pyrrolidone carboxylate. The additional moisturizing agent may be present in about 0.5% to about 15% by weight, such as 5% by weight, of the personal care formulation. The specific concentration of the additional moisturizing agent in the formulation may be adjusted based on various factors, such as the desired moisturizing effect, the stability of the formulation, the sensory properties of the formulation, or other characteristics.

In some aspects, the (first) moisturizing agent and the at least one additional (second) moisturizing agent may be present in a synergistically moisturizing effective ratio. This ratio may be determined based on the desired moisturizing effect of the formulation, the specific properties of the moisturizing agents, and other factors. In some cases, the ratio may be adjusted to optimize the moisturizing effect, the stability of the formulation, the sensory properties of the formulation, or other characteristics. For example, a higher ratio of the moisturizing agent to the additional moisturizing agent may provide a stronger moisturizing effect, while a lower ratio may result in a lighter, less greasy feel.

In some aspects, the personal care formulation may include at least one additional (second) moisturizing agent. This additional moisturizing agent may be selected from a group consisting of glycerol, urea, hydroxyalkylurea, lactic acid, sodium lactate, sodium pyrrolidone carboxylate, and combinations thereof. In some cases, the additional moisturizing agent may be specifically hydroxyethyl urea. The inclusion of an additional moisturizing agent may enhance the moisturizing properties of the formulation, providing additional benefits to the skin or hair.

In some embodiments, the personal care formulation may include one or more additives, such as solvent, thickener, film former, stabilizer, chelating agent, emollient, emulsifier, moisturizer, antioxidant, skin conditioning agent, preservative, pH adjuster, etc. A solvent may include aqua (water). Examples of thickeners include Acrylates/C10-30 Alkyl Acrylate Crosspolymer and Cetearyl Alcohol. Examples of emulsifiers include Cetearyl Alcohol and Ceteareth-20. Examples of emollients include Cetearyl Alcohol, Isoamyl Caprylate/Caprate, and Caprylic/Capric Triglyceride. Examples of chelating agents include Disodium EDTA and Citric Acid. Examples of preservatives include Phenoxyethanol and BHT. Examples of pH adjusters include sodium hydroxide and citric acid. Any of these additives may considered cosmetically acceptable vehicles, which refers to the base or carrier substance used in cosmetic formulations that delivers active ingredients to the skin or hair while ensuring the product is stable, effective, and pleasant to use. The one or more additive may be at least 0.1 wt %, such as 0.2 wt %, 0.5 wt %, 1 wt %, etc. of the personal care formulation.

A cosmetically acceptable vehicle in a cosmetic product may have the following properties: (1) Stabilizes—maintains the stability of active ingredients, preventing degradation or separation of the product; (2) Enhances application—improves the texture, spreadability, and absorption of the product on the skin or hair; (3) Non-irritating—safe, non-toxic, and non-sensitizing when used regularly on the skin or hair; and (4) Compatibility—do not interfere with the efficacy of the active ingredients in the formulation. Common examples of cosmetically acceptable vehicles include water (aqua), oils, emulsifiers, silicones, and certain polymers, each of which support the product's overall function and feel while carrying the active components to the target area.

In some embodiments, the personal care formulation may further comprise a thickener and an emulsifier, each present in at least 0.1% by weight of the personal care formulation. The thickener may serve to increase the viscosity of the formulation, improving its texture and spreadability. The emulsifier may help to stabilize the formulation, preventing the separation of its components and ensuring a uniform distribution of the moisturizing agent and the additional moisturizing agent throughout the formulation.

In some cases, the personal care formulation may further comprise at least one additive selected from the group consisting of sunscreen active agents, antiperspirant active agents, anti-acne agents, anti-dandruff agents, emollient agents, and combinations thereof. These additives may provide additional benefits to the skin or hair, enhancing the overall performance of the personal care formulation. For example, a sunscreen active agent may provide protection against UV radiation, an antiperspirant active agent may reduce perspiration, an anti-acne agent may help to prevent or treat acne, an anti-dandruff agent may help to control dandruff, and an emollient agent may provide additional moisturizing benefits. The specific type and amount of additive included in the formulation may depend on the desired properties and benefits of the formulation.

In some aspects, the personal care formulation may be specifically an anti-aging cream. This anti-aging cream may include a (first) moisturizing agent of formula R—COO—CH₂CH₂CH(CH₃)₂, where R is a linear or branched alkyl group of formula —(CH2)n-CH3, where n is 6 to 8, and at least one additional (second, third, etc.) moisturizing agent. The at least one additional moisturizing agent may have the formula R—COO—CH₂CH₂CH(CH₃)₂, where R is a linear or branched alkyl group of formula —(CH2)n-CH3, where n is different from that of the (first) moisturizing agent. The anti-aging cream may also include retinyl acetate as an active ingredient. Retinyl acetate is a form of vitamin A that is known for its anti-aging properties, such as reducing the appearance of fine lines and wrinkles, improving skin texture, and promoting skin cell turnover.

In some cases, the (first) moisturizing agent and the at least one additional (second, third, etc.) moisturizing agent may be present in the anti-aging cream in a synergistically moisturizing effective ratio. This ratio may be determined based on the desired moisturizing effect of the cream, the specific properties of the moisturizing agents, and other factors. For example, a higher ratio of the moisturizing agent to the additional moisturizing agent may provide a stronger moisturizing effect, while a lower ratio may result in a lighter, less greasy feel.

In some embodiments, the anti-aging cream may be formulated to enhance the skin permeation of retinyl acetate. The moisturizing agent may act as a penetration enhancer, helping to deliver retinyl acetate more effectively into the skin. This enhanced skin permeation may result in improved efficacy of the anti-aging cream, potentially leading to more noticeable anti-aging effects.

In some aspects, the anti-aging cream may be formulated for specific skin types or conditions. For example, the cream may be formulated for dry skin, oily skin, sensitive skin, mature skin, or other skin types or conditions. The cream may include additional ingredients that are beneficial for the specific skin type or condition, and the amount or type of moisturizing agent and retinyl acetate may be adjusted accordingly.

In some cases, the anti-aging cream may be applied to the skin using a variety of methods. For example, the cream may be applied using a finger, a brush, a sponge, a roller, a spray, or other suitable application tool. The specific method of application may depend on various factors, such as the form of the cream, the target area of application, and the desired evenness or precision of application.

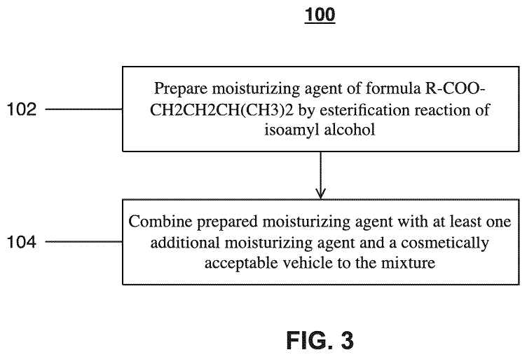

FIG. 3 illustrates a method 100 for creating a moisturizing composition. The method 100 includes two main steps: the synthesis of a moisturizing agent 102 and the combination of this agent with additional ingredients 104 to form the final personal care formulation.

In step 102, a moisturizing agent is prepared. The moisturizing agent is synthesized through an esterification reaction of an alcohol. The alcohol is reacted with a fatty acid to form the ester. The esterification reaction may be carried out in the presence of an acid catalyst and under conditions of heat to drive the reaction to completion. The resulting ester, which is the moisturizing agent, retains the alkyl group from the alcohol and the linear alkyl chain from the fatty acid.

Following the synthesis of the moisturizing agent, the method 100 proceeds to step 104. In this step, the moisturizing agent is combined with at least one additional moisturizing agent and a cosmetically acceptable vehicle to form the personal care formulation. The additional moisturizing agent may be any suitable agent known in the art for providing moisturizing benefits to the skin or hair. Non-limiting examples of additional moisturizing agents include glycerol, urea, hydroxyalkylurea, lactic acid, sodium lactate, and sodium pyrrolidone carboxylate. The cosmetically acceptable vehicle serves as a carrier for the moisturizing agents and can be any suitable vehicle known in the art for use in personal care formulations. The vehicle may be a liquid, semi-solid, or solid material that is safe for topical application and can facilitate the delivery of the moisturizing agents to the skin or hair.

In some aspects, the moisturizing agents are combined in a synergistically moisturizing effective ratio. This ratio may be determined based on the desired moisturizing effect of the formulation, the specific properties of the moisturizing agents, and other factors. In some cases, the ratio may be adjusted to optimize the moisturizing effect, the stability of the formulation, the sensory properties of the formulation, or other characteristics.

In some embodiments, the method 100 may further include additional steps, such as the addition of other ingredients to the formulation, the adjustment of the pH of the formulation, or the packaging of the formulation into suitable containers for storage and use. The specific steps and sequence of steps in the method 100 may vary depending on the specific requirements of the personal care formulation and the desired properties of the final product.

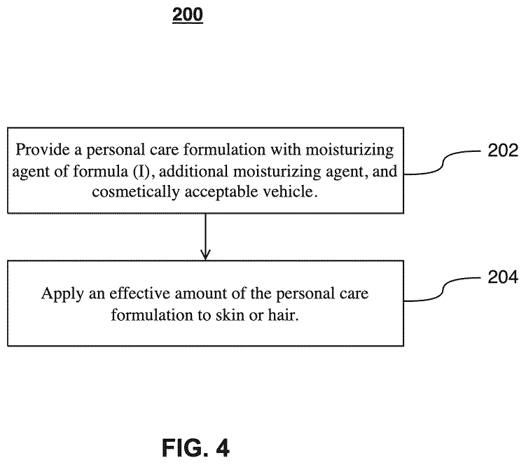

FIG. 4 illustrates a flowchart for a method 200 of applying a personal care formulation. The method 200 comprises two steps for moisturizing skin or hair. Step 202 involves providing a personal care formulation. This formulation may contain a (first) moisturizing agent, an additional (second) moisturizing agent, an active ingredient, and a cosmetically acceptable vehicle. Step 204 involves applying an effective amount of the personal care formulation to skin or hair.

In step 202, a personal care formulation is provided. The formulation may include a first moisturizing agent and a second moisturizing agent. The moisturizing agents may be present in a synergistically moisturizing effective ratio.

In some aspects, the personal care formulation may be provided in a variety of forms suitable for topical application to skin or hair. For example, the formulation may be provided as a cream, lotion, gel, serum, ointment, or other suitable form. The specific form of the formulation may depend on various factors, such as the desired application method, the target area of application, the specific skin or hair type, and the desired sensory properties of the formulation.

In step 104, an effective amount of the personal care formulation is applied to skin or hair. The effective amount may be determined based on the desired moisturizing effect, the specific properties of the moisturizing agents, and other factors. In some cases, the effective amount may be adjusted to optimize the moisturizing effect, the stability of the formulation, the sensory properties of the formulation, or other characteristics.

In some aspects, the personal care formulation may be applied to the skin or hair using a variety of methods. For example, the formulation may be applied using a finger, a brush, a sponge, a roller, a spray, or other suitable application tool. The specific method of application may depend on various factors, such as the form of the formulation, the target area of application, and the desired evenness or precision of application.

In some embodiments, the method 200 may further include additional steps, such as preparing the skin or hair for application, allowing the formulation to absorb or dry after application, or applying additional products or treatments before or after the formulation. The specific steps and sequence of steps in the method 200 may vary depending on the specific requirements of the personal care formulation and the desired properties of the final product.

The interactions between the active pharmaceutical ingredients (API), skin, and vehicle may determine: (1) the drug release, (2) the penetration through the stratum corneum (SC), and/or (3) the penetration through the viable skin layers. Drug release may refer to the process by which a drug is released from a delivery system into the body, where is may become available for absorption and therapeutic action. Drug release may include be characterized by rate of release, duration of release and/or amount of release. Factors to consider in the selection of an API include, for example, molecular weight (<600 Da); low melting point (<200° C.), which is related to appropriate solubility; a high but stable partition coefficient because very high partition coefficients may increase drug retention, thus inhibiting drug clearance from the skin, and/or solubility in water and oils to achieve a proper concentration gradient and increase the diffusion force over the skin. There may be a combination of multiple factors to guarantee the correct pathway for API into the skin.

The examples include the following steps of molecule synthesis, anti-aging cream formulation with retinyl acetate, cutaneous permeation methodology development to evaluate the cream permeation and the retinyl acetate permeation, and cream and retinyl acetate permeation quantification. These examples demonstrate the successful synthesis of Oxismooth® CP, its incorporation into anti-aging cream formulations, and its ability to enhance the cutaneous permeation of retinyl acetate, providing evidence of its potential as a novel, sustainable emollient for skincare applications.

### EXAMPLES

### Example 1: Synthesis of Oxismooth® CP

The synthesis of Oxismooth® CP, an ester emollient, was carried out using isoamyl alcohol and fatty acids, specifically octanoic (caprylic) acid and decanoic (capric) acid, on an industrial scale.

The synthesis process involved an esterification reaction, resulting in the formation of the ester emollient, as shown in Table 1.

TABLE 1

| | |
|---|---|
| Oxismooth ® CP | 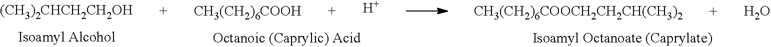 |
| | 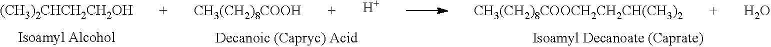 |

The product obtained had the following properties as shown in the Table 2:

TABLE 2

| Properties | Oxismooth ® CP |
|---|---|
| INCI Name | Isoamyl Caprylate/Caprate |
| CAS Number | 1365095-43-7 |
| Appearance @ 25° C. | Clear liquid |
| Gardner Color | 2 max. |
| Odor | Characteristic |
| Water, % | 0.75 max |
| Acid Value, mg KOH/g | 1.0 max |
| Saponification Value, mg KOH/g | 240.0-258.0 |
| Molecular Weight, g/mol | 227.13 |
| Density @ 20° C., g/cm³ | 0.859⁽¹⁾ |
| Boiling Point @ 760 mmHg, ° C. | 241-257 |
| Flash Point, ° C. | 96.5-133 |
| Refractive Index, 25° C. | 1.4260 |
| Pour Point, ° C. | <−20 |
| Viscosity, 25° C., BKF, mPa.s | 2.67 |
| Solubility in water | Partially soluble |
| Renewable Sources, % | 100 |
| Shelf-life | 24 months |

INCI stands for International Nomenclature of Cosmetic Ingredients. INCI is a standardized system for naming cosmetic ingredients that is used globally in the personal care and cosmetics industry. INCI names may be used on product labels and in formulation documentation to provide consistent, internationally recognized terminology for cosmetic ingredients. INCI names may be used to precisely identify the chemical components of the personal care formulation, such as “Isoamyl Caprylate/Caprate” for the moisturizing agent described. Using INCI names may ensure clarity and uniformity in describing the composition of the formulation, which may be crucial for regulatory compliance and effective communication within the industry.

CAS stands for Chemical Abstracts Service, a division of the American Chemical Society. CAS numbers are unique numerical identifiers assigned to chemical substances. The CAS number is provided for the moisturizing agent Isoamyl Caprylate/Caprate (CAS Number: 1365095-43-7). CAS numbers are widely used in scientific literature, databases, and regulatory documents to precisely identify chemical compounds, ensuring clarity and avoiding ambiguity when referring to specific substances. The inclusion of the CAS number may help definitively identify the chemical composition of the moisturizing agent.

Gardner Color refers to a standardized color scale used in the chemical and cosmetic industries to measure and describe the color of liquids, particularly oils and fats. The scale ranges from 1 to 18, with 1 being the lightest (almost colorless) and 18 being the darkest. In the context of the Oxismooth® CP product specification, a Gardner Color of 2 max indicates that the product is very light in color, nearly colorless. This low color value is desirable for cosmetic ingredients as it allows for greater flexibility in formulation without significantly impacting the final product's appearance. The Gardner Color test is performed using a colorimeter or spectrophotometer and is an important quality control parameter for ensuring consistency and purity in cosmetic and personal care ingredients.

A characteristic odor refers to a distinctive smell that is associated with a specific substance or material. The odor is often unique and can help in identifying or distinguishing that substance from others.

The saponification value is a parameter used to characterize oils, fats, and waxes in cosmetic and personal care formulations. The saponification value represents the number of milligrams of potassium hydroxide (KOH) required to saponify one gram of the sample. Saponification is the process by which triglycerides are hydrolyzed into fatty acids and glycerol in the presence of an alkali.

The term “hydrolyzed” refers to the chemical process of hydrolysis, which involves the breaking of chemical bonds through the addition of water. In the context of personal care formulations, hydrolyzed ingredients are often derived from larger molecules that have been broken down into smaller, more easily absorbed components. This process can enhance the performance and skin compatibility of certain ingredients. For example, hydrolyzed proteins are commonly used in hair and skin care products due to their ability to penetrate the hair shaft or skin barrier more effectively than their non-hydrolyzed counterparts. The hydrolysis process can also modify the properties of ingredients, potentially improving their solubility, stability, or functionality within a formulation. In the case of starches or other polysaccharides, hydrolysis can create smaller sugar molecules or oligosaccharides that may have different moisturizing or texturizing properties compared to the original ingredient.

For Oxismooth® CP, the saponification value range of 240.0-258.0 mg KOH/g indicates the amount of KOH needed to completely saponify one gram of the ester. This value provides information about the average molecular weight and chain length of the fatty acids in the ester. Saponification values typically vary inversely with the fatty acid chain length. A higher saponification value generally corresponds to shorter chain fatty acids, while a lower value indicates longer chain fatty acids.

The specified range for Oxismooth CP is consistent with esters derived from medium-chain fatty acids like caprylic (C8) and capric (C10) acids. This saponification value range helps ensure the consistency and quality of the product, as well as its suitability for use in personal care formulations where specific emollient and moisturizing properties are desired.

Molecular Weight, g/mol refers to the mass of one mole of a substance, expressed in grams. For isoamyl caprylate (caprate), the molecular weight is 227.13 g/mol. This value represents the sum of the atomic weights of all atoms in the molecule. The molecular weight is a characteristic that may influence properties, such as boiling point, melting point, and solubility. In the context of personal care formulations, the molecular weight can affect how the moisturizing agent interacts with the skin or hair, its ability to penetrate the stratum corneum, and its overall efficacy in providing moisturizing benefits. A relatively low molecular weight, such as 227.13 g/mol, may contribute to the moisturizing agent's ability to penetrate the skin barrier and provide effective hydration.

Density @20° C., g/cm³ refers to the mass per unit volume of the moisturizing agent at a temperature of 20 degrees Celsius, measured in grams per cubic centimeter. The property may be used to characterize the physical properties of the moisturizing agent and ensure consistency in formulation. The density value of 0.859 g/cm³ for Oxismooth® CP indicates that it is slightly less dense than water (which has a density of 1 g/cm³). This lower density may contribute to the light feel of the moisturizing agent when applied to skin. The density also affects how the moisturizing agent interacts with other ingredients in the personal care formulation, influencing factors such as mixing, stability, and texture of the final product.

The boiling point at 760 mmHg (standard atmospheric pressure) is a physical property that provides information about the volatility and thermal stability of the moisturizing agent. For Oxismooth® CP, the boiling point range of 241-257° C. indicates that the compound has a relatively high boiling point, which is typical for esters derived from medium-chain fatty acids and higher alcohols. The high boiling point contributes to the stability of the moisturizing agent in personal care formulations, ensuring that it does not easily evaporate during storage or application. The boiling point range, rather than a single value, reflects the fact that Oxismooth® CP is a mixture of isoamyl caprylate and isoamyl caprate, each with slightly different boiling points. This property is relevant for formulation considerations, as it can affect the behavior of the moisturizing agent during processing and in the final product, particularly in terms of its ability to remain stable and effective in various personal care applications.

Flash Point, ° C. refers to the lowest temperature at which the vapors of a substance can ignite when exposed to an ignition source. For Oxismooth® CP, the flash point range is 96.5-133° C. This relatively high flash point indicates that the moisturizing agent has low volatility and is not easily flammable at room temperature, which is an important safety consideration for personal care formulations. The flash point is a crucial parameter for handling, storage, and transportation of the ingredient, as it can inform proper safety precautions and regulatory compliance. In the context of personal care products, a higher flash point generally indicates greater stability and safety during normal use conditions. The wide range provided (96.5-133° C.) likely accounts for variations in the exact composition of the Oxismooth® CP, which is a mixture of isoamyl caprylate and isoamyl caprate.

Refractive Index, 25° C. refers to the ratio of the speed of light in a vacuum to its speed in the moisturizing agent at a temperature of 25 degrees Celsius. For Oxismooth® CP, the refractive index is 1.4260. The property may be important in personal care formulations as it can affect the appearance and optical properties of the product. A refractive index close to that of the skin (approximately 1.44) can contribute to a more natural look when the formulation is applied. The refractive index can also influence the formulation's ability to scatter light, which may impact its visual effects on the skin, such as reducing the appearance of fine lines or imperfections. In the context of emollients, the refractive index can affect how the product interacts with light on the skin surface, potentially contributing to a smoother or more radiant appearance.

Pour Point refers to the lowest temperature at which a liquid will flow when cooled under specified conditions. For the Oxismooth® CP product, the pour point is listed as <−20° C., indicating that the product may remain in a liquid state and flow at temperatures below −20° C. The low pour point may be advantageous for personal care formulations, as it can ensure the product remains in a usable liquid form even at very low temperatures. The property may be used to help maintain the stability and performance of the formulation during storage and transportation in cold climates. The low pour point may also contribute to the product's versatility, allowing it to be incorporated into a wide range of personal care formulations without concerns about solidification or changes in viscosity at low temperatures.

Viscosity is a measure of a fluid's resistance to flow. In the context of personal care formulations, viscosity is a property that affects the texture, spreadability, and overall performance of the product. For Oxismooth® CP, the viscosity is measured at 25° C. using a Brookfield viscometer (BKF). The value of 2.67 mPa·s indicates that Oxismooth® CP has a relatively low viscosity, which contributes to its light, non-greasy feel and easy spreadability when used in personal care formulations. This low viscosity is advantageous for creating formulations with a pleasant texture and good absorption characteristics. The viscosity of an emollient can significantly impact the sensory properties of the final product, influencing factors such as how easily it can be applied to the skin, how quickly it absorbs, and the overall skin feel after application.

The solubility in water of the moisturizing agent is a property that can affect the moisturizing agent's behavior in personal care formulations and its interactions with the skin. For Oxismooth® CP, the solubility is described as “Partially soluble” in water. Partial solubility indicates that the moisturizing agent has both hydrophilic and lipophilic characteristics, which can be advantageous in personal care applications. The partial water solubility can allow the moisturizing agent to interact with both the aqueous and oil phases of an emulsion, potentially enhancing its ability to stabilize formulations and deliver moisturizing benefits to the skin. Additionally, the partial water solubility may contribute to the moisturizing agent's ability to form a protective barrier on the skin surface while still allowing for some water retention, which can help maintain skin hydration. The balance between water solubility and oil solubility may be crucial for the moisturizing agent's effectiveness in personal care formulations, as it can allow for optimal interaction with both the skin's lipid barrier and its natural moisture content.

### Example 2: Formulation of Anti-Aging Creams

Two anti-aging cream formulations were prepared, one using Oxismooth® CP (Sample DM415) and the other using a comparative emollient (Sample JL270). Both formulations contained 1% retinyl acetate as the active ingredient. The detailed composition of the formulations is provided in Table 3.

TABLE 3

| | | | | Sample | Sample |
|---|---|---|---|---|---|
| | | | | DM415 | JL 270 |
| | | | | With | With |
| | | | | Indorama | Benchmark |
| | | | | emollient | emollient |
| | | | | % in | % in |
| Phase | Components | INCI Name | Supplier | Actives | Actives |
| A | Deionized | Aqua (water) | — | qsp 100 | qsp 100 |
| | water | ACRYLATES/C1 | Volp | 0.5 | 0.5 |
| | Carbopol | 0- 30 ALKYL | | | |
| | FD 2020 | ACRYLATE | | | |
| | | CROSSPOLYMER | | | |
| | Disodium | DISODIUM | Poly | 0.1 | 0.1 |
| | EDTA | EDTA | commercial | | |
| B | Alkonat | Cetearyl | Indorama | 7 | 7 |
| | 1618 C30 p | alcohol | | | |
| | Alkonat | Ceteareth-20 | Indorama | 7 | 7 |
| | CE 200 f | | | | |
| | Oxismooth | Isoamyl | Indorama | 5 | 0 |
| | CP | caprylate/caprate | | | |
| | Caprylic/capric | Caprylic/capric | Engenharia | 0 | 5 |
| | triglyceride | triglyceride | | | |
| | BHT | BHT | Synth | 0.1 | 0.1 |
| C | Retinyl acetate | Retinyl acetate | ELEVE | 1 | 1 |
| | Phenoxyethanol | Phenoxyethanol | Indorama | 1 | 1 |
| | (glysolv ephl) | | | | |
| D | Sodium | Sodium hydroxide | Neon | qsp pH | qsp pH |
| | hydroxide | | | 6.3 +/− 0.2 | 6.3 +/− 0.2 |
| | Citric Acid | CITRIC ACID | Synth | qsp pH | qsp pH |
| | | | | 6.3 +/− 0.2 | 6.3 +/− 0.2 |

Aqua (water) refers to purified water used as a solvent and base ingredient in personal care formulations. It serves as the primary vehicle for dissolving and dispersing other ingredients in the formulation. Water is essential for maintaining proper viscosity, texture, and stability of the product. In the context of this personal care formulation, water likely constitutes the bulk of the composition, providing a medium for the moisturizing agents and other components to interact and deliver their benefits to the skin or hair. The use of purified water ensures that the formulation is free from contaminants and impurities that could affect its safety, efficacy, or stability.

ACRYLATES/C10-30 ALKYL ACRYLATE CROSSPOLYMER is a synthetic polymer used as a thickening agent, emulsion stabilizer, and rheology modifier in personal care formulations. It is formed by crosslinking acrylates copolymer with a long-chain alkyl acrylate, typically containing 10 to 30 carbon atoms in the alkyl group. This crosslinking creates a three-dimensional network structure that gives the polymer its unique properties. In the context of the personal care formulation described, ACRYLATES/C10-30 ALKYL ACRYLATE CROSSPOLYMER serves several important functions: (1) Thickening—It increases the viscosity of the formulation, improving its texture and spreadability; (2) Emulsion stabilization—The polymer helps maintain the stability of the oil-in-water emulsion by preventing the separation of oil and water phases; (3) Rheology modification—It provides the formulation with specific flow characteristics, which can affect how the product feels when applied to the skin and how it spreads; and (4) Suspension—It can help keep other ingredients, such as particulates or oil droplets, evenly dispersed throughout the formulation. The inclusion of this ingredient in the formulation at 0.5% by weight suggests that it plays a crucial role in achieving the desired texture and stability of the anti-aging cream. Its ability to form a gel network in aqueous systems makes it particularly useful in creating formulations that have a smooth, non-greasy feel while still providing effective delivery of active ingredients like retinyl acetate.

DISODIUM EDTA, also known as disodium ethylenediaminetetraacetic acid, is a chelating agent commonly used in personal care formulations. It functions as a preservative enhancer and stabilizer by binding to metal ions that could otherwise catalyze oxidation reactions or promote microbial growth. In the context of this personal care formulation, DISODIUM EDTA helps to maintain the stability and efficacy of other ingredients, particularly antioxidants and preservatives. It may also contribute to the overall texture and feel of the product by preventing the formation of precipitates or discoloration that could result from metal ion interactions. The inclusion of DISODIUM EDTA at 0.1% by weight suggests that it plays a supportive role in maintaining the quality and shelf life of the anti-aging cream formulation.

Cetearyl alcohol is a mixture of fatty alcohols, primarily consisting of cetyl alcohol and stearyl alcohol. It is commonly used in personal care formulations as an emollient, emulsifier, and thickening agent. In the context of this personal care formulation, cetearyl alcohol likely serves multiple functions:

1. Emollient: It helps to soften and smooth the skin, providing a pleasant feel to the formulation.

2. Emulsifier: It aids in stabilizing the oil-in-water emulsion of the cream, helping to prevent separation of the oil and water phases.

3. Thickener: It contributes to the viscosity and texture of the formulation, giving it a desirable consistency.

4. Conditioning agent: In hair care products, it can act as a conditioning agent, helping to improve the feel and manageability of hair.

The inclusion of cetearyl alcohol at 7% by weight in both Sample DM415 and Sample JL270 suggests that it plays a significant role in the formulation's texture, stability, and skin feel. Its presence may contribute to the overall moisturizing effect of the personal care formulation and may also influence the permeation of other ingredients, such as the moisturizing agent and retinyl acetate, through the skin.

Ceteareth-20 is a nonionic surfactant and emulsifier commonly used in personal care formulations. It is derived from cetearyl alcohol and ethylene oxide. In the context of this personal care formulation, ceteareth-20 serves several important functions:

1. Emulsification: It helps to stabilize the oil-in-water emulsion by reducing the surface tension between the oil and water phases, preventing separation of the formulation.

2. Solubilization: Ceteareth-20 can help solubilize other ingredients, improving the overall stability and homogeneity of the formulation.

3. Texture enhancement: It contributes to the smooth, creamy texture of the formulation, improving its spreadability and skin feel.

4. Moisturization: While primarily an emulsifier, ceteareth-20 can also provide some moisturizing benefits to the skin.

The inclusion of ceteareth-20 at 7% by weight in both Sample DM415 and Sample JL270 suggests that it plays a significant role in the formulation's stability, texture, and overall performance. Its presence may also influence the permeation of other ingredients, such as the moisturizing agent and retinyl acetate, through the skin by altering the formulation's interaction with the stratum corneum.

Isoamyl caprylate/caprate refers to the moisturizing agent used in the personal care formulation described in this patent application. It is an ester compound formed by the reaction of isoamyl alcohol with a mixture of caprylic (C8) and capric (C10) fatty acids. This compound has the general formula R—COO—CH2CH2CH(CH3)2, where R is a linear alkyl group of formula —(CH2)n-CH3, and n is 6 or 8.

The isoamyl portion (—CH2CH2CH(CH3)2) is derived from isoamyl alcohol, which is obtained from sugar cane through a sustainable process. The caprylate/caprate portion (R—COO—) comes from the fatty acids, typically sourced from coconut or palm kernel oil. This combination results in a 100% renewable, plant-derived emollient.

Isoamyl caprylate/caprate, also known by the trade name Oxismooth® CP, serves as the primary moisturizing agent in the formulation. It provides several benefits, including improved spreadability, fast absorption, and a pleasant, non-greasy skin feel. The compound's unique structure allows it to enhance the permeation of active ingredients, such as retinyl acetate, through the skin, potentially improving the efficacy of anti-aging formulations.

Caprylic/capric triglyceride is a mixed ester derived from caprylic acid, capric acid, and glycerin. It is commonly used as an emollient and skin-conditioning agent in personal care formulations. This ingredient is known for its light, non-greasy feel and its ability to spread easily on the skin. In the context of this personal care formulation, caprylic/capric triglyceride serves as the benchmark emollient against which the performance of Oxismooth CP (isoamyl caprylate/caprate) is compared. The inclusion of caprylic/capric triglyceride at 5% by weight in Sample JL270 allows for a direct comparison with the Oxismooth CP in Sample DM415, which is also present at 5% by weight. This comparison is crucial for evaluating the relative efficacy of Oxismooth CP as a novel, sustainable emollient in enhancing the skin permeation of active ingredients like retinyl acetate.

BHT, or butylated hydroxytoluene, is a synthetic antioxidant commonly used in personal care formulations to prevent oxidation and extend the shelf life of products. In the context of this personal care formulation, BHT likely serves as a preservative and stabilizer, helping to maintain the efficacy of other ingredients, particularly those susceptible to oxidation such as retinyl acetate. The inclusion of BHT at 0.1% by weight in both Sample DM415 and Sample JL270 suggests that it plays a crucial role in maintaining the stability and quality of the anti-aging cream formulation over time. By preventing oxidation, BHT helps to ensure that the active ingredients remain effective throughout the product's intended shelf life.

Retinyl acetate is a form of vitamin A commonly used in skincare formulations, particularly in anti-aging products. It is an ester of retinol (vitamin A) and acetic acid. In the context of this personal care formulation, retinyl acetate serves as the active ingredient, present at 1% concentration in both the Sample DM415 and Sample JL270 formulations. Retinyl acetate is known for its ability to promote skin cell turnover, stimulate collagen production, and improve skin texture and tone. It is often preferred in cosmetic formulations due to its stability and lower potential for skin irritation compared to other retinoids. The inclusion of retinyl acetate in these formulations allows for the evaluation of how different emollients (Oxismooth CP and the benchmark emollient) affect the permeation and efficacy of this active ingredient through the reconstructed human epidermis (RHE) in the in vitro skin permeation studies.

Phenoxyethanol is a preservative commonly used in personal care formulations to prevent microbial growth and extend the shelf life of products. In the context of this personal care formulation, phenoxyethanol likely serves as a broad-spectrum antimicrobial agent, helping to protect the formulation from bacterial and fungal contamination. It is effective against a wide range of microorganisms and is often preferred in cosmetic formulations due to its mild nature and low potential for skin irritation compared to some other preservatives. The inclusion of phenoxyethanol at 1% by weight in both Sample DM415 and Sample JL270 suggests that it plays a crucial role in maintaining the stability and safety of the anti-aging cream formulation over time. Its presence helps ensure that the active ingredients, including retinyl acetate, remain effective and that the product remains safe for use throughout its intended shelf life.

Sodium hydroxide, also known as lye or caustic soda, is a strong alkaline compound with the chemical formula NaOH. In the context of this personal care formulation, sodium hydroxide likely serves as a pH adjuster. It is used to increase the pH of the formulation, making it more alkaline. This pH adjustment is crucial for maintaining the stability and efficacy of other ingredients in the formulation, particularly the active ingredients like retinyl acetate. The proper pH can also affect the overall performance of the product on the skin, including its moisturizing capabilities and potential for irritation. Sodium hydroxide is typically used in very small amounts in personal care products, as indicated by the “qsp” (quantum satis pro, or as much as is needed) notation in the formulation table. The precise amount is adjusted to achieve the desired pH of 6.3±0.2, which is slightly acidic to neutral and generally compatible with skin pH. It's important to note that while sodium hydroxide is a strong base, its concentration in the final formulation is carefully controlled to ensure product safety and efficacy.

Citric acid is an organic acid commonly used in personal care formulations as a pH adjuster and preservative enhancer. In the context of this personal care formulation, citric acid likely serves to fine-tune the pH of the product to the desired level of 6.3±0.2. This slightly acidic pH is generally compatible with the skin's natural pH and helps maintain the stability and efficacy of other ingredients in the formulation, particularly active ingredients like retinyl acetate. Citric acid can also act as a mild exfoliant, helping to improve skin texture and tone. Additionally, it may function as a chelating agent, binding to metal ions that could otherwise destabilize the formulation or reduce its shelf life. The use of citric acid in combination with sodium hydroxide allows for precise pH adjustment, ensuring that the final product has the optimal pH for skin compatibility and ingredient stability. The “qsp” (quantum satis pro, or as much as is needed) notation in the formulation table indicates that the amount of citric acid used is adjusted as necessary to achieve the target pH.

The formulations were prepared by heating and mixing the ingredients in phases A and B separately, then combining them under agitation to form an oil-in-water emulsion. The emulsion was then cooled, and the ingredients of phases C and D were added. The final formulation was filled under an inert atmosphere into aluminum collapsible tubes and stored below 20° C. The specifics of preparing the formulations JL270 and DM415 are shown in Table 4.

TABLE 4

| Sequence | Description |
|---|---|
| 1 | Mix and heat ingredients of Phase A to 75-80° C. |
| | Heat ingredients of Phase B to 75-80° C. |
| 2 | Under agitation (1000 rpm), pour Phase B to Phase A. |
| | Homogenize the emulsion. |
| | Cool down to 40° C., under agitation. |
| 3 | Add Phase C. |
| | Homogenize and cool down the emulsion to room temperature |
| | while stirring. |
| 4 | Adjust pH with ingredients of Phase D if necessary. |
| 5 | The finished formulation should be filled under an inert |
| | atmosphere into aluminum collapsible tubes. |
| | Keep samples below 20° C. during storage and transport. |

### Example 3: In Vitro Skin Permeation Studies

A cutaneous permeation methodology was developed to evaluate the cream permeation and the retinyl acetate permeation. The methodology developed considered the recommendations of Guide N^{o} 428 OECD6.

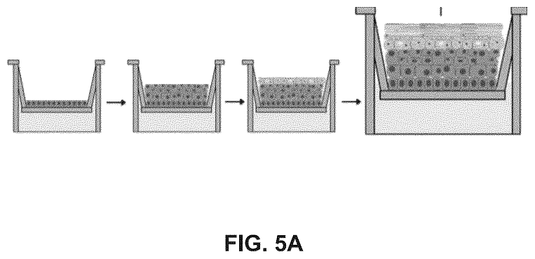

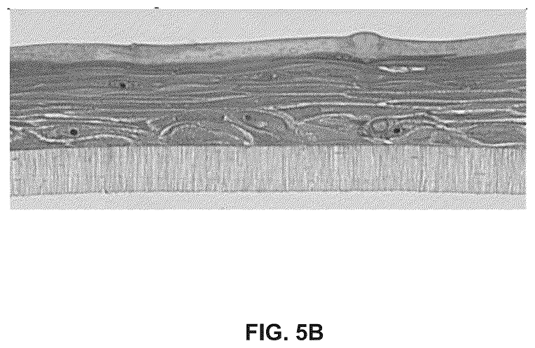

The tests were conducted in vitro using a reconstructed human skin (RHE), produced by ELEVE Science. The produced RHE has a quality specification that guarantee the quality and reproducibility of the RHE produced as cell viability, number of cells layers and integrity of epidermis barrier function. FIG. 5A shows a schematic model of skin growth for reconstruction of the human skin. FIG. 5B shows the microscopy image of the RHE produced by ELEVE Science. The image was obtained using HE staining (Hematoxylin-Eosin), a technique used in histology to visualize tissues under the microscope.

FIG. 5A illustrates a four-stage process of cell growth and proliferation in a laboratory culture dish. This process is used to create the reconstructed human epidermis (RHE) that is used in the skin permeation studies described in the present disclosure. In the first stage, a small number of cells are seeded at the bottom of the dish, appearing as a thin layer. In the second stage, the cells divide and grow, with the layer becoming thicker and more densely populated. In the third stage, the cells continue to proliferate, expanding upwards and filling more of the dish volume. In the final stage, the cells have fully populated the dish, forming a thick, multilayered structure that reaches near the top of the container. Throughout the process, the culture medium, represented by the pink color, decreases in volume as it is consumed by the growing cell population. The container remains constant in size across all stages, providing a stable environment for cell growth.

In some aspects, the RHE used in the skin permeation studies may be produced using a similar process of cell growth and proliferation. The RHE may be grown in a laboratory culture dish or other suitable container, with the cells seeded at the bottom of the dish and allowed to divide and grow to form a multilayered structure. The culture medium may be periodically replenished to provide the necessary nutrients for cell growth. The resulting RHE may closely mimic the structure and properties of natural human epidermis, making it a suitable model for evaluating the skin permeation of the personal care formulation.

In some cases, the RHE may be produced using cells derived from human skin tissue. The cells may be obtained, for example, from skin biopsies or other sources of human skin tissue. In some embodiments, the cells used to produce the RHE may be keratinocytes, the primary cell type found in the epidermis. Other types of skin cells, such as melanocytes or Langerhans cells, may also be included in the RHE to more closely mimic the composition of natural human epidermis.

In some embodiments, the RHE may be produced using a different process or technique. For example, the RHE may be produced using a 3D bioprinting technique, in which layers of cells are printed onto a substrate to form a multilayered structure. Alternatively, the RHE may be produced using a tissue engineering technique, in which cells are seeded onto a biodegradable scaffold and allowed to proliferate and form a multilayered structure. The specific process or technique used to produce the RHE may depend on various factors, such as the desired properties of the RHE, the available resources, and the specific requirements of the skin permeation studies.

Referring to FIG. 5B, the figure shows a microscopic cross-section of ELEVE Science RHE, which resembles human skin tissue. The image is stained in shades of pink and purple, which is typical of histological preparations. The top layer shows a thin, stratified structure that likely represents the epidermis, with several distinct cellular layers visible. Beneath this is a thicker layer with a more fibrous appearance, which is likely the dermis. This dermal layer contains numerous elongated structures running parallel to the skin surface, which may represent collagen fibers. At the bottom of the image is a lighter-colored layer with a more uniform appearance, which could represent subcutaneous tissue. This microscopic view of skin structure illustrates the layers through which topical skincare formulations, such as those described in the patent, must penetrate to deliver active ingredients and provide moisturizing effects.

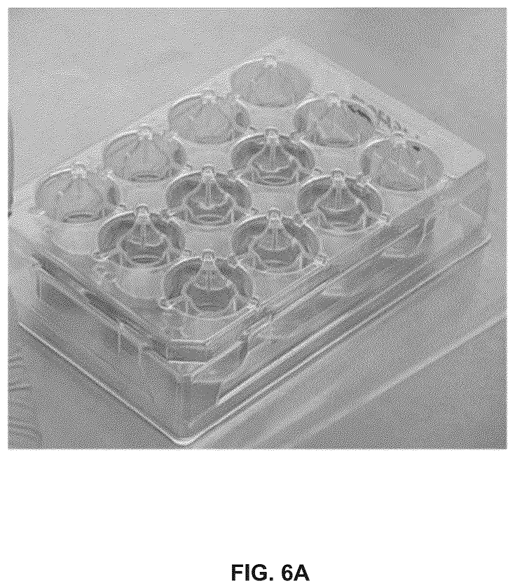

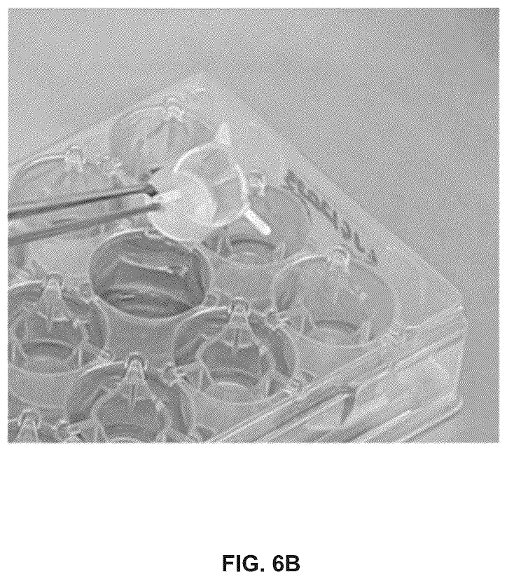

FIGS. 6A and 6B show the matrix used on the experiments. Each matrix has twelve different channels where each one is used for one single in-vitro test. Each RHE is composed of one receptor channel where the sample is applied, an RHE, and a receptor solution to receive the emollient and/or active ingredient permeated through the RHE. A receptor solution refers to the solution on the side of the permeation apparatus (e.g., a Franz diffusion cell) that collects the permeated substance (such as an emollient or active ingredient) as it passes through a membrane or skin model. The receptor solution typically mimics physiological conditions to simulate how the substance would behave in the body. FIG. 6A shows a matrix of 12 RHE for in vitro permeation tests, and FIG. 6B shows the detail of one single cell used for each experiment (ELEVE Science, n.d.-b).

Referring to FIG. 6A, the figure illustrates a multi-well plate or tray used in laboratory settings for conducting experiments or tests on small volumes of liquid samples. In the context of the present disclosure, this multi-well plate is used for conducting in vitro skin permeation studies to evaluate the personal care formulations containing the moisturizing agent or mixture of moisturizing agents. The wells of the multi-well plate are filled with reconstructed human epidermis (RHE) samples, and the personal care formulations are applied to these samples. The varying shades of pink in the wells may indicate different concentrations or compositions of the personal care formulations being tested. For example, a darker shade of pink may represent a higher concentration of the moisturizing agent or a different ratio of the first moisturizing agent to the second moisturizing agent. In some cases, the varying shades of pink may also indicate the extent of permeation of the first moisturizing agent and the second moisturizing agent into the RHE samples over time.

In some aspects, the multi-well plate may include more or fewer wells than shown in FIG. 6A, depending on the number of samples or conditions being tested. For example, a larger multi-well plate with more wells may be used to test a wider range of concentrations or compositions of the personal care formulations, or to test the formulations under different conditions (e.g., different temperatures or pH levels). Conversely, a smaller multi-well plate with fewer wells may be used for preliminary screening tests or for testing a smaller number of samples or conditions.

In some embodiments, the multi-well plate may be made of a material that is compatible with the personal care formulations and the RHE samples, and that does not interfere with the skin permeation studies. Suitable materials for the multi-well plate may include, for example, plastic, glass, or other non-reactive materials. The multi-well plate may also be transparent or semi-transparent to allow for visual inspection of the wells and the samples therein.

In some cases, the multi-well plate may be used in conjunction with other laboratory equipment or devices to facilitate the skin permeation studies. For example, the multi-well plate may be placed in an incubator to maintain a controlled temperature and humidity environment for the studies. The multi-well plate may also be used with a microscope or other imaging device to visually observe and record the permeation of the moisturizing agent and the additional moisturizing agent into the RHE samples.

Referring to FIG. 6B, the figure illustrates a multi-well plate setup used in the in vitro skin permeation experiments described in the present disclosure. Each well of the multi-well plate contains a reconstructed human epidermis (RHE) sample, which serves as a model for human skin in the permeation studies. The personal care formulations containing the moisturizing agent and the at least one additional moisturizing agent are applied to these RHE samples. The multi-well plate setup allows for multiple samples to be tested simultaneously under the same conditions, facilitating the comparison of different formulations or different concentrations of the same formulation.

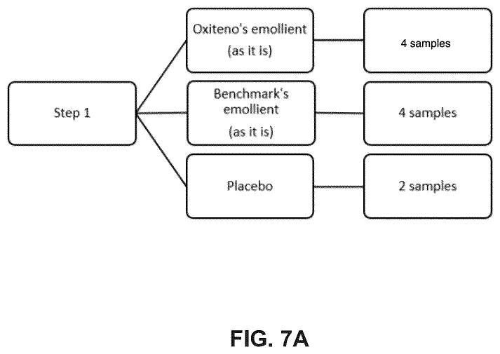

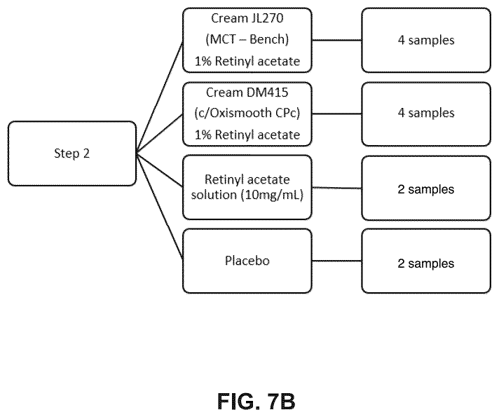

The experimental procedure for in vitro skin permeation was conducted in two different steps as shown in FIGS. 7A and 7B. FIG. 7A shows step 1 for evaluating Oxiteno's emollient and Benchmark's emollient without the active ingredient. FIG. 7B shows step 2 for evaluating Oxiteno's emollient and Benchmark's emollient with the anti-aging formulation containing retinyl acetate compared to a retinyl acetate solution. The dermal permeation study in RHE was performed under optimized conditions according to OECD10, using an RHE model with 1.13 cm² of diffusion area and a volume of 4.00 mL in the receiving compartment.

FIG. 7A illustrates an experimental design system for evaluating emollients. Experiments conducted in step 1 were realized without the active ingredient in order to detect the emollient absorption on the RHE. In vitro skin permeation test was performed on the first step experiments using 10 cells from
the 12 available on the RHE matrix, divided as following:
4 cells evaluating Oxismooth CP emollient, 4 cells evaluating Benchmark emollient, and 2 cells used as blank (“placebo”).

This layout allows for a comparative analysis between Oxiteno's emollient, a benchmark emollient, and a placebo control. The system is designed to facilitate the evaluation of different emollients by providing a structured approach to sample distribution and testing. The use of multiple samples for each emollient type enhances the statistical reliability of the experimental results. The inclusion of a placebo group with fewer samples serves as a control for the experiment.

In some aspects, the experimental design system may be used to evaluate the skin permeation properties of different emollients. For example, the system may be used to compare the skin permeation of the Oxiteno moisturizing agent, with that of a benchmark emollient. The system may also be used to evaluate the skin permeation of a placebo, which may serve as a control for the experiments.

In some cases, the experimental design system may include more or fewer branches than shown in FIG. 7A, depending on the number of emollients or conditions being tested. For example, the system may include additional branches for testing different concentrations or compositions of the moisturizing agent, or for testing the emollient under different conditions (e.g., different temperatures or pH levels).

In some embodiments, the experimental design system may be used in conjunction with other laboratory equipment or devices to facilitate the skin permeation studies. For example, the system may be used with a multi-well plate or other suitable container for holding the reconstructed human epidermis (RHE) samples. The system may also be used with a microscope or other imaging device to visually observe and record the permeation of the emollient into the RHE samples.

In some aspects, the experimental design system may be used to evaluate the synergistic moisturizing effect of the moisturizing agent and the at least one additional moisturizing agent. For example, the system may be used to compare the skin permeation of a personal care formulation containing the (first) moisturizing agent and the additional (second) moisturizing agent with that of a formulation containing only the (first) moisturizing agent or only the additional (second) moisturizing agent. This could potentially provide valuable information about the synergistic moisturizing effect of the two agents when combined in a synergistically moisturizing effective ratio.

FIG. 7B illustrates the experimental design for Step 2 of a study comparing different formulations containing retinyl acetate. Experiments conducted in step 2 were realized with samples prepared with addition of 1% retinyl acetate to evaluate the retinyl acetate permeation on RHE in comparison with the benchmark product.

In step 2 experiments, one new RHE matrix was used and all 12 cells available were used and were divided as follows: 4 cells for evaluating Sample DM415 (antiaging cream formulated using Oxismooth CP and 1% retinyl acetate), 4 cells for evaluating Sample JL 270 (antiaging cream formulated using Benchmark and 1% retinyl acetate), 2 cells for evaluating retinyl acetate solution (1% active), and 2 cells used as blank cells (“placebo”).

In some aspects, the experimental design may include more or fewer samples for each sample type, depending on the specific requirements of the skin permeation studies. For example, a larger number of samples may be used to increase the statistical power of the study, while a smaller number of samples may be used for preliminary screening tests or for testing a smaller number of conditions.

In some cases, the experimental design may include additional sample types not shown in FIG. 7B. For example, the design may include samples of other anti-aging creams formulated with different emollients or different concentrations of retinyl acetate. The design may also include samples of other types of personal care formulations, such as lotions, serums, or gels, that contain the first moisturizing agent and the second moisturizing agent.

In some embodiments, the experimental design may include additional steps or procedures not shown in FIG. 7B. For example, the design may include steps for preparing the reconstructed human epidermis (RHE) samples, steps for applying the personal care formulations to the RHE samples, and steps for measuring the permeation of the first moisturizing agent and the second moisturizing agent into and through the RHE samples. The specific steps and procedures included in the experimental design may depend on various factors, such as the desired properties of the personal care formulation, the specific requirements of the skin permeation studies, and the available resources and equipment.

All single samples evaluated followed the procedure below:
a) Application of a finite dose (5 mg/cm²) of each sample over the RHE.
b) Six hours of contact between the sample and RHE.
c) Receptor solution collected and evaluated every one hour.
d) After six hours, the RHE surface was washed ten times using 10 mL of DPBS (phosphate-buffered saline solution) to ensure the complete removal of sample that was not permeated. The sample that was removed because it was not permeated was not quantified.
e) Final quantification:
a. Sample permeated on the RHE was bioanalyzed using GC/MS and reported in μg/cm².
b. Sample permeated through RHE and reaching the receptor solution was bioanalyzed using GC/MS and reported in μg/cm².

The studies were conducted in two steps. In the first step, the permeation of the emollients alone (Oxismooth® CP and the benchmark emollient) was evaluated. In the second step, the permeation of the anti-aging cream formulations containing retinyl acetate was evaluated.

The results of the first step indicated that the benchmark emollient permeated the RHE to a greater extent (approximately 190 μg/cm²) compared to Oxismooth® CP (approximately 85 μg/cm²).

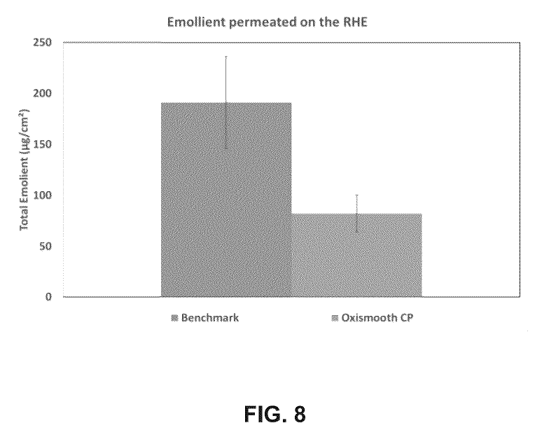

FIG. 8 illustrates a bar graph comparing the total emollient permeated on the reconstructed human epidermis (RHE) for the Benchmark product (sum of capric and caprylic acid) and Oxismooth® CP (isoamyl caprate and caprylate). The y-axis represents the total emollient permeated in μg/cm², while the x-axis shows the two products being compared. The Benchmark product, represented by an orange bar, shows a higher level of emollient permeation at approximately 190 μg/cm². In contrast, Oxismooth® CP, represented by a gray bar, shows a lower level of emollient permeation at about 85 μg/cm². Both bars have error bars, with the Benchmark product showing a larger margin of error. This graph visually demonstrates the difference in emollient permeation between the two products when applied to reconstructed human epidermis.

FIG. 8 presents the total amount of emollients permeated into the RHE. Although they presented a great difference in terms of emollient permeation, in terms of retinyl acetate permeation, it does not guarantee the permeation of the active ingredient (retinyl acetate) because the permeation of the active ingredient (retinyl acetate) is a result of the interaction of emollient, active ingredient, and skin.

The permeation performance may be affected by different emollients performance mainly due to differences in physical state, molecular weight, lipophilicity, ionization, and interaction with the skin membrane. Considering the main emollients differences as shown in Table 4, the higher benchmark permeation, can be attributed for a combination of its higher lipophilicity and higher viscosity.

In some aspects, the difference in emollient permeation between the Benchmark product and Oxismooth® CP may be attributed to the different chemical structures and properties of the emollients. For example, the Benchmark emollient may have a higher molecular weight or a different degree of lipophilicity compared to Oxismooth® CP, which could affect its ability to penetrate the stratum corneum and be retained in the RHE. The Benchmark emollient may also have a different solubility profile in the cosmetically acceptable vehicle used in the personal care formulation, which could affect its release from the formulation and its subsequent permeation into the RHE. The properties of Oxismooth® CP and Benchmark emollient are shown in Table 5.

TABLE 5

| Property | Oxismooth ® CP | Benchmark |
|---|---|---|
| Chemical | Isoamyl | Caprylic/Capric Triglyceride |
| composition | Caprylate/Caprate | |
| Molecular | 227 | 372 |
| weight | | |
| Viscosity | 2.67 | 32.3 |
| Lop P | Partially water soluble | Water insoluble log P > 8 |

In some cases, the lower level of emollient permeation observed for Oxismooth® CP may be advantageous for certain applications. For example, a lower level of emollient permeation may result in a slower release of the emollient from the personal care formulation, providing a sustained moisturizing effect over a longer period of time. A lower level of emollient permeation may also reduce the potential for skin irritation or sensitivity reactions that can sometimes occur with high concentrations of emollients.

In some embodiments, the personal care formulation may be formulated to optimize the permeation of the (first) moisturizing agent and the at least one additional (second, third, etc.) moisturizing agent through the stratum corneum and into the RHE. For example, the formulation may include ingredients that enhance intercellular penetration, such as penetration enhancers or solvents. Alternatively, the formulation may be designed to enhance follicular penetration, for example by including ingredients that increase the solubility of the agents in sebum, the oily substance present in hair follicles. In some cases, the formulation may be designed to enhance transcellular penetration, for example by including ingredients that increase the permeability of skin cell membranes.

In the second step, the accumulated amount of retinyl acetate in the receptor solution over time was measured for the two formulations and a control sample of retinyl acetate solution. The results showed that the formulation containing Oxismooth® CP (Sample DM415) demonstrated a higher accumulated amount of retinyl acetate in the receptor solution over time compared to the benchmark formulation (Sample JL270) and the control sample. This suggests that Oxismooth CP enhances the skin permeation of retinyl acetate, potentially leading to improved efficacy of the anti-aging cream.

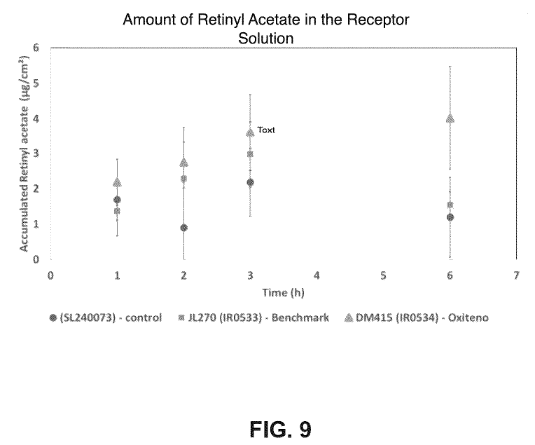

FIG. 9 illustrates a graph showing the accumulated amount of retinyl acetate in a receptor solution over time. The graph compares three different samples: a control (SL240073), a benchmark (JL270), and a sample labeled DM415 (IR0534)—Oxiteno. Each sample is represented by a different colored and shaped data point (blue circles, orange squares, and gray triangles respectively). The graph shows measurements taken at 1, 2, 3, and 6 hour intervals. Error bars are included for each data point.

Although no emollient permeation was detected on the receptor solution on the first step experiments, when the active retinyl acetate was added on the antiaging formulation, both emollients were able to carry the active to the RHE and also to reach the receptor solution.

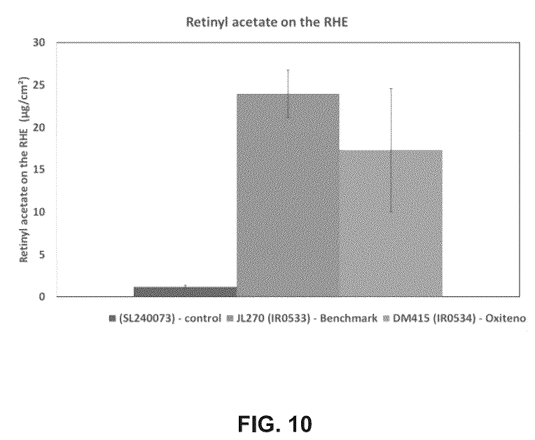

FIG. 9 reports the accumulated amount of retinyl acetate in the receptor solution after six 20 hours of anti-aging cream formulation in contact with the RHE. Oxismooth® CP sample promoted a better retinyl acetate permeation than the Benchmark sample; both samples enabled the retinyl acetate permeation through RHE to the receptor solution. FIG. 9 shows the retinyl acetate accumulated on the RHE at the end of the six hours of experiments and shows retinyl acetate absorption into the RHE by the Oxismooth® CP sample, suggesting the importance of emollient presence on the performance of the active permeation of the active ingredient retinyl acetate since the control sample, composed of a retinyl acetate solution presented a low permeation on the RHE. Also see control results in FIG. 10. It appears that less retinyl acetate from the benchmark and control were accumulated on the RHE at about 6 hours than at about 3 hours. At 6 hours, the amount of retinyl acetate from Oxiteno emollient accumulated in RHE was at least steady, if not more.

In some aspects, the graph provides a visual representation of the skin permeation capabilities of the personal care formulation containing the (first) moisturizing agent and the at least one additional (second, third, etc.) moisturizing agent. The graph shows that the sample DM415, which contains the moisturizing agent and retinyl acetate, demonstrates a higher accumulated amount of retinyl acetate in the receptor solution over time compared to the benchmark and control samples. This suggests that the moisturizing agent may enhance the skin permeation of retinyl acetate, potentially leading to improved efficacy of the personal care formulation.

FIG. 10 illustrates a bar graph comparing the accumulated amount of retinyl acetate on the reconstructed human epidermis (RHE) for three different samples: a control sample (SL240073), a benchmark sample (JL270—IR0533), and a sample labeled DM415 (IR0534)—Oxiteno. The y-axis represents the accumulated retinyl acetate in μg/cm², while the x-axis shows the three different samples. The control sample, represented by a blue bar, shows a very low concentration of retinyl acetate, indicating minimal permeation of retinyl acetate into the RHE. The benchmark sample, represented by an orange bar, shows a higher concentration of retinyl acetate, indicating a greater degree of permeation into the RHE. The Oxiteno sample, represented by a gray bar, shows a moderate concentration of retinyl acetate, indicating a level of permeation into the RHE that is between that of the control and benchmark samples. Error bars are included for each sample, indicating the range of variability in the measurements.

In some aspects, the bar graph in FIG. 10 provides a visual representation of the skin permeation capabilities of the personal care formulation containing the moisturizing agent or mixture of moisturizing agent. The graph shows that the sample DM415, which contains the moisturizing agent and retinyl acetate, demonstrates a higher accumulated amount of retinyl acetate in the RHE compared to the benchmark and control samples. This suggests that the moisturizing agent may enhance the skin permeation of retinyl acetate, potentially leading to improved efficacy of the personal care formulation.

The extensive work promoted a clear evidence of positive synergy of emollients with retinyl acetate active permeation in the RHE, enhancing a better active absorption on the skin when compared with the retinyl acetate application in solution (control). Consequently, suggesting a better cost dosage antiaging formulation and performance, avoiding the active ingredient (retinyl acetate) to be washed out after its application. Moreover, Oxismooth® CP proved a better performance than the benchmark product when used to carrier the active retinyl acetate through the RHE.

The following clauses illustrated example subject matter described herein.

CLAUSE 1. A personal care formulation comprising: a mixture of a first moisturizing agent and a second moisturizing agent, the first moisturizing agent having a formula R—COO—C₅H₁₁, wherein R is a linear or branched alkyl group of formula —(CH₂)ₙ—CH₃ and n is 6; and the second moisturizing agent having a formula R—COO—C₅H₁₁, wherein R is a linear or branched alkyl group of formula —(CH₂)ₙ—CH₃ and n is 8; the mixture of the first moisturizing agent and the second moisturizing agent exhibiting greater permeation in a reconstructed human epidermis than an emollient comprising caprylic triglyceride and capric triglyceride.

CLAUSE 2. The personal care formulation of CLAUSE 1, further comprising retinyl acetate, the retinyl acetate exhibiting greater permeation in a reconstructed human epidermis than a personal care formulation comprising retinyl acetate and an emollient comprising caprylic triglyceride and capric triglyceride.

CLAUSE 3. The personal care formulation of CLAUSES 1 or 2, further comprising retinyl acetate, the retinyl acetate exhibiting continued or increased permeation in a reconstructed human epidermis after about 6 hours of exposure to the reconstructed human epidermis.

While the disclosure has been described in connection with what is presently considered to be the most practical and preferred embodiments, it is to be understood that the disclosure is not to be limited to the disclosed embodiments, but on the contrary, is intended to cover various modifications and equivalent arrangements included within the spirit and scope of the appended claims, which scope is to be accorded the broadest interpretation so as to encompass all such modifications and equivalent structures as permitted under the law. Furthermore, it should be understood that while the use of the word preferable, preferably, or preferred in the description above indicates that feature so described may be more desirable, it nonetheless may not be necessary and any embodiment lacking the same may be contemplated as within the scope of the disclosure, that scope being defined by the claims that follow. In reading the claims it is intended that when words such as “a,” “an,” “at least one” and “at least a portion” are used, there is no intention to limit the claim to only one item unless specifically stated to the contrary in the claim. Further, when the language “at least a portion” and/or “a portion” is used the item may include a portion and/or the entire item unless specifically stated to the contrary.

What is claimed is:

1. A personal care formulation comprising:
a mixture of a first moisturizing agent and a second moisturizing agent;
the first moisturizing agent having a formula R—COO—C₅H₁₁, wherein R is a linear or branched alkyl group of formula —(CH₂)ₙ—CH₃ and n is 6;
the second moisturizing agent having a formula R—COO—C₅H₁₁, wherein R is a linear or branched alkyl group of formula —(CH₂)ₙ—CH₃, where n is 8; and
a cosmetically acceptable vehicle.

2. The personal care formulation of claim 1, further comprising retinyl acetate.

3. The personal care formulation of claim 1, wherein the first moisturizing agent comprises isoamyl caprylate.

4. The personal care formulation of claim 1, wherein the second moisturizing agent comprises isoamyl caprate.

5. The personal care formulation of claim 1, wherein the first moisturizing agent is about 0.5% to about 15% by weight of the personal care formulation.

6. The personal care formulation of claim 1, wherein the second moisturizing agent is about 0.5% to about 15% by weight of the personal care formulation.

7. The personal care formulation of claim 1, further comprising at least one additive selected from the group consisting of thickeners, emulsifiers, emollients, chelating agents, preservatives, antioxidants, pH adjusters, and combinations thereof.

8. A method of moisturizing skin or hair, comprising:
applying to skin or hair an effective amount of a personal care formulation comprising
a mixture of a first moisturizing agent and a second moisturizing agent;
the first moisturizing agent having a formula R—COO—C₅H₁₁, wherein R is a linear or branched alkyl group of formula —(CH₂)ₙ—CH₃ and n is 6;
the second moisturizing agent having a formula R—COO—C₅H₁₁, wherein R is a linear or branched alkyl group of formula —(CH₂)ₙ—CH₃, where n is 8; and
a cosmetically acceptable vehicle.

9. The personal care formulation of claim 8, further comprising retinyl acetate.

10. The personal care formulation of claim 8, wherein the first moisturizing agent comprises isoamyl caprylate.

11. The personal care formulation of claim 8, wherein the second moisturizing agent comprises isoamyl caprate.

12. The personal care formulation of claim 8, wherein the first moisturizing agent is about 0.5% to about 15% by weight of the personal care formulation.

13. The personal care formulation of claim 8, wherein the second moisturizing agent is about 0.5% to about 15% by weight of the personal care formulation.

14. The personal care formulation of claim 8, further comprising at least one additive selected from the group consisting of thickeners, emulsifiers, emollients, chelating agents, preservatives, antioxidants, pH adjusters, and combinations thereof.

15. A process for preparing a personal care formulation, comprising:
preparing a mixture of a first moisturizing agent and a second moisturizing agent by an esterification reaction of an alcohol derived from sugar cane and a fatty acid derived from a vegetal source, the first moisturizing agent having a formula R—COO—C₅H₁₁, wherein R is a linear or branched alkyl group of formula —(CH₂)ₙ—CH₃ and n is 6; the second moisturizing agent having a formula R—COO—C₅H₁₁, wherein R is a linear or branched alkyl group of formula —(CH₂)ₙ—CH₃, where n is 8; and
combining the mixture of the first moisturizing agent and the second moisturizing agent with a cosmetically acceptable vehicle.

16. The personal care formulation of claim 15, further comprising retinyl acetate.

17. The personal care formulation of claim 15, wherein the first moisturizing agent comprises isoamyl caprylate.

18. The personal care formulation of claim 15, wherein the second moisturizing agent comprises isoamyl caprate.

19. The personal care formulation of claim 15, wherein the first moisturizing agent is about 0.5% to about 15% by weight of the personal care formulation, and the second moisturizing agent is about 0.5% to about 15% by weight of the personal care formulation.

20. The personal care formulation of claim 15, further comprising at least one additive selected from the group consisting of thickeners, emulsifiers, emollients, chelating agents, preservatives, antioxidants, pH adjusters, and combinations thereof.
